(19) United States
(12) Patent Application Publication
(10) Pub. No.: US 2017/0014637 A1
(43) Pub. Date: Jan. 19, 2017

(54) SYSTEM WITH AN ELECTROMAGENTIC FIELD GENERATOR WITH COILS FOR TREATING TUMORS AND A METHOD FOR TREATING TISSUE

(71) Applicant: The United States of America, as represented by the Secretary, Department of Health and Human Serv, Bethesda, MD (US)

(72) Inventor: Peter J. Basser, Washington, DC (US)

(73) Assignee: The United States of America, as represented by the Secretary, Department of Health and Human Serv, Bethesda, MD (US)

(21) Appl. No.: 15/125,079

(22) PCT Filed: Mar. 17, 2015
(86) PCT No.: PCT/US2015/021066

§ 371 Date: Sep. 9, 2016

Related U.S. Application Data
(60) Provisional application No. 61/954,494, filed on Mar. 17, 2014.

(51) Int. Cl. A61N 2/02; A61N 1/40; A61N 2/00

(52) U.S. Cl. CPC A61N 2/02; A61N 2/006; A61N 1/40

(57) ABSTRACT

Treatment apparatus includes a plurality of coils configured to generate time-varying magnetic fields that induce electric fields within a subject. In one example, electric field strengths of at least 1 V/cm are produced in brain tissues exhibiting Glioblastoma Multiforme (GBM). Fields are applied based on computer-assisted modeling using electromagnetic characteristics of the brain, and tissue locations identified as exhibiting disease using imaging data. A head mounted assembly of coils can be used for convenient, portable treatment.

### FIELD

The disclosure pertains to treatment of Glioblastoma Multiforme or other cancers using induced electromagnetic fields.

### BACKGROUND

Treatment of cancer in the brain presents significant challenges. It can be difficult to administer medications in an effective manner that permits therapeutic amounts to reach brain areas requiring treatment. In other cases, surgical or other mechanical interventions may be precluded due to the location of the diseased area. For example, access to the diseased area may be possible only by unacceptable injury to other brain areas. Conventional treatments based on surgery and chemotherapy are not effective in achieving long-term patient survival.

Treatment of Glioblastoma Multiforma (GBM), the most common and lethal primary brain cancer, has been previously proposed based on electric fields applied with an array of capacitively coupled electrodes placed on a patient's scalp. Oscillating electric fields at frequencies of 100-300 kHz and magnitudes of 1-3 V/cm have been demonstrated to disrupt mitotic division in GBM cells in culture. Such tumor treating fields (TTFs) have shown promise in the treatment of GBM. One significant problem with this approach is the difficulty in accurately targeting the necessary electric fields to diseased tissues. Both the electrodes and currents needed for treatment may impact patient comfort. Currently, the patient's hair must be shaven and electrodes must be affixed to the skin. The high currents required to penetrate the scalp and skull into the brain parenchyma often result in skin rashes. In some cases, currents intended for treatment may be shunted away from the diseased tissues. Thus, despite significant advances in chemotherapy, surgical procedures, and TTF-based treatments, alternative approaches are still needed.

### SUMMARY

According to some examples, systems comprise a plurality of coils and an electromagnetic field generator that energizes the plurality of coils so as to apply a tumor treating field (TTF) to a specimen region. The TTF has a time-varying magnetic field component that induces an electric field of magnitude of at least 0.1 V/cm at a frequency of between 50 kHz and 500 kHz. In some embodiments, the specimen region is associated with a tumor, and in particular examples, at least one brain tumor. In some alternatives, the electromagnetic field generator energizes the plurality of coils so as to apply the TTF to the specimen region along a plurality of axes. In other examples, the electromagnetic field generator sequentially energizes the plurality of coils so as to sequentially apply the TTF to the specimen region along at least two axes. According to other embodiments, the electromagnetic field generator energizes the plurality of coils so that the TTF is applied at a plurality of frequencies between 50 kHz and 500 kHz. In one example, a rigid shell is shaped to at least partially enclose the specimen region, wherein the plurality of coils is secured to the rigid shell. In still other examples, the coils of the plurality of coils are flexibly interconnected so as to be wrappable about the specimen region. In yet other alternatives, the coils are energized by the electromagnetic field generator so as to produce a time-varying magnetic field that produces an induced electric field magnitude of at least 0.5, 1.0, 2, 5, 10, or 100 V/cm.

Methods comprise identifying a tissue to be exposed to a treatment electromagnetic field. The treatment electromagnetic field is applied to at least a portion of the tissue, the treatment electromagnetic field having a time-varying magnetic field component that produces an induced electric field having an effective magnitude of at least 0.5 V/cm at a frequency of between about 50 kHz and 500 Hz in the region to be exposed. In some cases, the applied treatment electromagnetic field is based on electrical characteristics of the tissue determined from an image of the tissue. In other examples, the treatment electromagnetic field is applied along at least two axes. In one example, tissue is at least a portion of a patient brain. In still other alternatives, the treatment electromagnetic field is applied along a plurality of axes and at a plurality of frequencies in the range of 50 kHz to 500 kHz. In some embodiments, the treatment electromagnetic field is applied so as to have an effective duration of at least 10 ms, 100 ms, 1 s, 10 s, 100 s, or 1000 s. In some cases, the treatment electromagnetic fields are applied sequentially or simultaneously at a plurality of frequencies, and differing coils or sets of coils can be switched so as to apply the treatment electromagnetic fields. In some examples, a swept frequency electromagnetic field is applied using one, two or more coils or coils sets that are sequentially or simultaneously energized. In particular examples, the treatment electromagnetic field is applied with coils that are fixed with respect to the tissue and the treatment electromagnetic field is applied along different axes by selectively energizing corresponding coils. In still other embodiments, the treatment electromagnetic field is determined based on an electrical model of the tissue that includes at least one tissue permittivity and at least one tissue conductivity.

At least one non-transitory computer readable medium comprises computer-executable instructions for a method of applying treatment electromagnetic fields to a tissue. The method comprises selecting a portion of the tissue exhibiting cancer and generating a sequence of electrical currents and coupling the sequence of electrical currents to at least one coil so as to produce the treatment electromagnetic fields in the selected portion of the tissue. Typically, the treatment electromagnetic field includes a time-varying magnetic field that induces a time-varying electric field having a magnitude of at least 0.1 V/cm. In other examples, the electrical currents have magnitudes such that the treatment electromagnetic fields have time varying magnetic field components that produce an induced electric field magnitude of at least 0.5, 1.0, 2, 5, 10, or 100 V/cm. In some examples, the treatment electromagnetic fields are applied along a least two axes and the electrical currents are selected based on a sequence of electrical currents that includes electrical currents at at least two frequencies between 50 kHz and 1 MHz. In still further examples, the tissue is a portion of the brain is identified as exhibiting GBM. In other alternatives, methods further comprise estimating an electromagnetic field distribution in the tissue based on a tissue image and at least one tissue permittivity and at least one tissue conductivity, wherein the sequence of electrical currents is based on the estimated electromagnetic field distribution. In some embodiments, the tissue image is an MR image.

The foregoing and other features and advantages of the disclosed technology will become more apparent from the following detailed description, which proceeds with reference to the accompanying figures.

### BRIEF DESCRIPTION OF THE DRAWINGS

FIG. 1 is a block diagram of a representative system for applying transcranial electromagnetic fields.

FIG. 2 illustrates a representative method of applying transcranial electromagnetic fields.

FIGS. 3A-3B illustrate a representative apparatus arranged to apply magnetic fields to selected regions of interest with a plurality of coils.

FIG. 4 illustrates a representative apparatus arranged to secure a plurality of transcranial electromagnetic field generators to a helmet.

FIG. 5 illustrates a representative treatment apparatus that includes a transceiver configured to communicate with a mobile computing device.

FIG. 6 illustrates an alternative head-mounted treatment apparatus.

FIG. 7 illustrates a user interface for providing treatment data for determination of drive signals for producing treatment fields.

FIG. 8 illustrates a representative computing environment for implementation of the disclosed methods and apparatus.

FIG. 9 illustrates a representative mobile computing device configured for use in conjunction with determination and application of treatment fields.

FIG. 10 illustrates cloud-based provisioning of treatment fields.

FIG. 11 illustrates a representative coil assembly.

FIG. 12 illustrates exposure of a specimen region along multiple axes using associated coils.

FIG. 13 illustrates a planned exposure method.

### DETAILED DESCRIPTION

As used in this application and in the claims, the singular forms “a,” “an,” and “the” include the plural forms unless the context clearly dictates otherwise. Additionally, the term “includes” means “comprises.” Further, the term “coupled” does not exclude the presence of intermediate elements between the coupled items.

The systems, apparatus, and methods described herein should not be construed as limiting in any way. Instead, the present disclosure is directed toward all novel and non-obvious features and aspects of the various disclosed embodiments, alone and in various combinations and sub-combinations with one another. The disclosed systems, methods, and apparatus are not limited to any specific aspect or feature or combinations thereof, nor do the disclosed systems, methods, and apparatus require that any one or more specific advantages be present or problems be solved. Any theories of operation are to facilitate explanation, but the disclosed systems, methods, and apparatus are not limited to such theories of operation.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed systems, methods, and apparatus can be used in conjunction with other systems, methods, and apparatus. Additionally, the description sometimes uses terms like “produce” and “provide” to describe the disclosed methods. These terms are high-level abstractions of the actual operations that are performed. The actual operations that correspond to these terms will vary depending on the particular implementation and are readily discernible by one of ordinary skill in the art.

In some examples, values, procedures, or apparatus' are referred to as “lowest”, “best”, “minimum,” or the like. It will be appreciated that such descriptions are intended to indicate that a selection among many used functional alternatives can be made, and such selections need not be better, smaller, or otherwise preferable to other selections.

Some examples below are described with reference to Glioblastoma Multiforme (GBM), a form of brain cancer. However, the disclosed methods and apparatus can be configured for use in other applications as well.

The disclosed methods and apparatus generally provide moderate frequency electric fields or tumor treating fields (TTFs) sufficient to interfere with mitosis or possibly other cellular and extracellular processes that allow cancer cells to divide and grow. The disclosed methods and apparatus generally target rapidly dividing cells. TTFs are generally applied at frequencies of a few kHz and typically do not produce neural stimulation as a neural stimulation threshold rises dramatically with frequency, so that at TTF (kHz) frequencies, neurons tend not to fire. As used herein, TTF refers to an electric and/or magnetic field at frequencies between 1 kHz and 1 MHz, 2 kHZ and 1 MHz, 50 kHz and 1 MHz, 100 kHz and 1 MHz, 10 kHz and 1 MHz, 20 kHZ and 1 MHz, 50 kHz and 750 kHz, and 50 kHz and 500 MHz. Higher frequency fields are generally less useful due to, in some cases, lesser penetration into tissues, tissue heating, or tissue ablation. Lower frequency fields tend to produce neural and muscular stimulation. However, in some applications, such lower or higher frequency fields can be used. In addition, TTFs are generally associated with electric field strengths of between about 1-3 V/cm, but lesser or greater field strengths can be used, typically at least about 0.01 V/cm, 0.1 V/cm, 0.2 V/cm, or 0.5 V/cm. In some cases, portable or wearable apparatus that produce TTFs permit convenient, long-term exposure to TTFs so that high field strengths are not necessary for effective treatment. Induced AC fields in the kHz range can be produced with current-carrying coils placed over the scalp—outside the brain—to generate TTFs. In general, current-carrying coils can be placed outside of the body to induce electric fields in a selected region of the body. While not required, in some examples, specialized circuits can be used for energy storage are used, to reduce weight and size of treatment devices, improving portability and ease of use, and in some cases, making treatment apparatus wearable.

In an alternating electric field, charges and polar molecules are subjected to forces of alternating direction so that ionic flows and dipole rotate. These oscillations may hamper mitosis by interfering with formation of a normal functioning mitotic spindle as tubulin units may align with an applied electric field rather than the filament axis. Polymerization of tubulin subunits is necessary for the formation of functional mitotic spindles (microtubules) that are essential for the successful completion of mitosis. This could also explain the mitotic arrest of TTF-treated cells.

Cellular morphology during cytokinesis resembles an hourglass shape as the two daughter cells are forming. In the presence of TTFs, a non-uniform intracellular electric field is created which is characterized by a higher field intensity at the furrow between the dividing cells. This non-uniform field exerts unidirectional forces on polarizable charged macromolecules and organelles, a process termed dielectrophoresis. Those forces could interfere with spindle tubulin orientation and the dielectrophoretic (DEP) force could induce particles toward or away from the furrow. Cytoplasmatic components and organelles may pile up at the furrow within a few minutes, interfering with cytokinesis and possibly leading to incomplete cell division and eventual cell destruction. These proposed physical mechanisms are likely mechanisms for producing an anti-mitotic effect but other mechanisms electric-field mediated mechanisms may also cause this anti-mitotic effect. However, the disclosed methods and apparatus do not require such physical mechanisms, and these proposed mechanisms are provided only to illustrate some possibilities. The claimed methods and apparatus are not to be limited by any such physical mechanisms.

While the disclosed examples are generally described with reference to treatment of brain cancer, such induced TTFs can be used to treat other cancers in the body, such as in the torso (lungs) and even in deep tissue. For example, similar methods and apparatus can use specialized coils intended for different body areas, such as the neck, torso or limbs. In some cases, coils for different areas are substantially the same, but multiple coils are provided in different arrangements and are secured so as to be suited for placement at a selected body location. Some applications include treatment of human melanoma, glioma, cancers of the lung, prostate, kidney, pancreas, and breast, mouse adenocarcinoma and melanoma, and rat glioma, and treatment of similar diseases in humans or in veterinary applications. In addition, the disclosed methods and apparatus can be used in other applications in which cell division, cell proliferation, tumor growth, and/or angiogenesis is to be inhibited or disrupted. Disruption of angiogenesis could be useful in controlling or treating so-called wet macular degeneration. Rapid growth of neurons in the brain may be related to autism, and disruption or hindering of cell division may be applicable during development as a means to reduce local cell proliferation. Application of TTFs is associated with prolonged and abnormal mitosis in vitro, and cell death subsequent to cytokinesis.

In general, induced TTFs permit exposure and/or treatment of selected specimen regions so as to interfere with cell division. TTF exposures can be advantageous in that longer duration, higher field exposures can be obtained by limiting exposure to a selected region. In addition, targeting exposures to a specific region tends to reduce any unwanted effects associated with exposure.

Several mechanisms may be responsible for the anti-mitotic effect produced by exposure to TTFs. TTFs are associated with bidirectional ion flows and oscillations of dipoles. TTFs can interfere with formation of a normal functioning mitotic spindle as tubulin subunits are forced to align with an applied field rather than with a filament axis. Thus, their main function during mitosis, which is to generate the spatially organized microtubule spindle by precise choreographed alignment, is interrupted by external electric fields, since the tubulin subunits are forced to align with the impressed electric field rather than with the filament axis. Polymerization of tubulin subunits is necessary for formation of functional mitotic spindles (microtubules) that are essential for the successful completion of mitosis. This effect could also explain the mitotic arrest of TTF-treated cells.

Alternatively, TTFs may be associated with a dielectrophoretic effect. Cellular morphology during cytokinesis resembles an hourglass shape as the two daughter cells are forming. In the presence of TTFs, a non-uniform intracellular electric field is created which is characterized by a higher field intensity at a furrow between the dividing cells. It is believed that this non-uniform field exerts unidirectional forces on polarizable macromolecules and organelles, a process termed dielectrophoresis. Those forces interfere with spindle tubulin orientation and it was speculated that the DEP force moves particles toward the furrow. Cytoplasmatic organelles would accumulate at the cleavage furrow within a few minutes, interfering with cytokinesis and possibly leading to cell destruction.

The above-noted and other responses of cells to induced electric fields depend on tissue and cell structures, including orientation, dielectric constants, and conductivities. For example, permittivity and conductivity of tumor cells tend to be higher than surrounding tissues due to their higher water content. Glial cells can have very different dielectric properties, and induced field strength and frequency can be selected based on such specimen characteristics.

As disclosed herein, various types of imaging apparatus can be configured to produce specimen images based on specimen interrogation with acoustic waves, photons, electromagnetic radiation, or application of electric and/or magnetic fields. As used herein, an image refers to a viewable image of a specimen or a portion thereof as well as a stored representation of an image. Stored images can be in one or more computer-readable media as an image file in a JPEG or other format. Images can be stored locally (i.e., at a location near the specimen) or stored for retrieval via a local area network or a wide area network such as the internet. For convenience, examples based on two dimensional images are described, but in other examples, one dimensional (line) images, single point images, or three dimensional images can be used.

In the disclosed examples, time-varying magnetic fields are used to induce electric currents (eddy currents) in conductive specimens of interest. A significant application is to produce eddy currents in the brain or other organs, referred to herein as electromagnetically induced Tumor Treating Field (TTF) therapy. Image information and electromagnetic modeling can be used to identify specimen/subject characteristics for a particular specimen/subject and determine exposure characteristics such as induced field strengths, coil currents, frequency, field orientation(s).

The distribution of electromagnetically induced Tumor Treating Field (TTF) therapy can be predicted and optimized using various mathematical and computational modeling means, such as the Finite Element Method (FEM). Such models can incorporate coil geometry and properties, as well as head morphology and geometry, and head electromagnetic properties to predict the distribution of induced electric field and currents within the brain or other tissue.

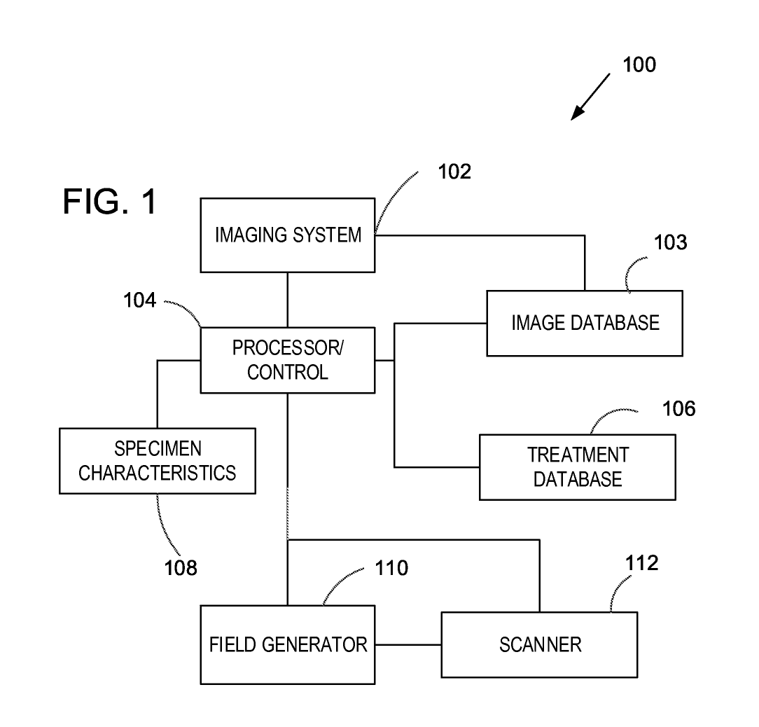

Referring to FIG. 1, a system for applying fields to a specimen or patient includes an imaging system 102 that is arranged so as to produce an image of a selected portion of a specimen. For example, one or more brain areas of a patient can be imaged to identify regions to which application of fields can be used for treatment. The imaging system can be an X-ray system such as an X-ray computed tomography (X-ray CT) system, a positron emission tomography (PET) imaging system, an ultrasonic imager, a single-photon emission computed tomography (SPECT) system, an MRI system, or other imaging system that produces images suitable for specimen evaluation. Typically, the imaging system stores a specimen image in an image database in a memory 103 or other storage device. A processor/controller 104 is coupled to the memory 103 so as to access one or more stored images. In some examples, then processor 104 is configured to identify areas of the specimen for treatment based on one or more image characteristics. However, in other examples, the imaging system 102 is configured to provide an indication of particular specimen areas for treatment. Such areas can be noted as highlighted in a stored image (for example, as colored or light/dark areas as displayed), or a table or other listing of specimen areas can be stored as well. Identification can be based on clinician evaluation or an automated image evaluation process. In some cases, treatment locations are provided to the processor/controller 104 as a series of image coordinates, or as defined treatment volumes.

With areas for treatment identified by the processor/controller 104, appropriate exposure fields are selected based on characteristics stored in a treatment database 106. The database 106 can store field magnitudes, durations pulse shapes, repetition rates, pulse periods, pulse frequencies or frequency spreads, field type, interpulse durations, or other pulse characteristics. The treatment database can store such parameters for a plurality of specimen conditions so that suitable values can be selected for a particular specimen and specimen condition. For example, a specimen database 108 can store specimen characteristics such as specimen conductivity and magnetic permeability, density, composition, orientation, or other features as a function of location. Specimen characteristics can be used by the processor/controller 104 in determining exposure fields to compensate for field attenuation or enhancement due to local specimen properties. Local variations in density, composition, or specimen anisotropies can be used to modify applied field characteristics so that intended fields tend to reach targeted specimen locations. In some examples, the field generator 110 can be controlled to vary a position of an intended field by application of suitable electrical signals to one or more devices such as coils. By selectively activating a plurality of field coils, a specimen area can be exposed to an intended field without requiring field coil or specimen movement. However, a scanning system 112 can be provided to produce relative movement between field coils and a specimen to facilitate treatment of extended specimen regions.

In treatment of GBM, MRI or X-ray CT imaging systems can be used to identify lesions that can be associated with GBM tissues. More reliable identification of GBM lesions generally is based on biopsy results. In some cases, perfusion or diffusion MRI and MR spectroscopic measurements of metabolite concentrations can be used as well. Surgical removal of GBM tissues can be used, with or without tumor contrast enhancement using a fluorescent dye such as 5-aminolevulinic acid. These procedures also permit collection of location data for subsequent use in defining electromagnetic field exposures. Suitable stereotactic procedures can also be used to locate areas for treatment.

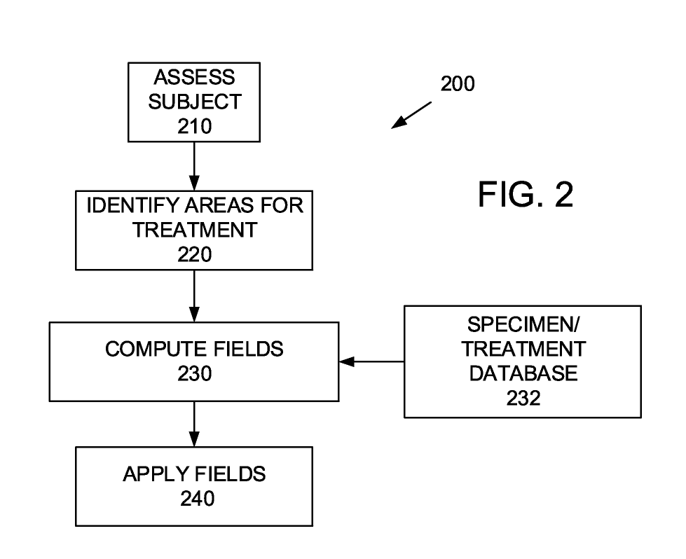

A representative method 200 is illustrated in FIG. 2. At 210, a subject is assessed based on, for example, one or more subject images or other subject evaluations. Based on the subject assessment, at 220 one or more subject regions are identified for treatment. The identified regions can be associated with diseased tissues such as malignancies, or other regions for which treatment is deemed appropriate. At 230, field characteristics such as pulse duration or field magnitude are selected for application to the identified regions. Different field characteristics can be identified for some or all regions. Typically, preferred treatment conditions can be provided based on parameters associated with tissue characteristics, disease type, region location in the subject and other characteristics that are stored in a database 232. At 240, the fields are applied.

Various configurations of magnetic fields can be applied. Field frequencies of between 1 Hz and 100 MHz, 10 Hz and 10 MHz. 100 Hz and 1 MHz, 1 kHz and 1 MHz, 50 kHz and 1 MHz, 50 kHz and 500 kHz, 100 kHz and 500 kHz, and 100 kHz and 300 kHz are typically used. Magnetic field amplitudes (and rates of change) are selected to produce treatment electric field strengths of between 0.1 V/cm and 100 V/cm, 0.5 V/cm and 20 V/cm, 1 V/cm and 10 V/cm, or 1 V/cm and 3 V/cm. Magnetic fields selected to produce electric field magnitudes of at least 0.5, 1, 2, or 5 V/cm are generally preferred for effective disruption of mitotic division processes in GBM or other cancers. Coupling of time-varying magnetic fields into the brain (including deep brain areas) tends not to be limited by conductive pathways that can shunt applied fields away from a selected target region. Direct contact with the scalp, such as low resistance electrical contact with conductive gels or other materials, is not required. TTF generating coils can be placed comfortably around the scalp without coil-to-skin contact required or the patient's hair to be shaven. Induced electric fields can more readily provide whole brain coverage, are more readily steered in both direction and distribution.

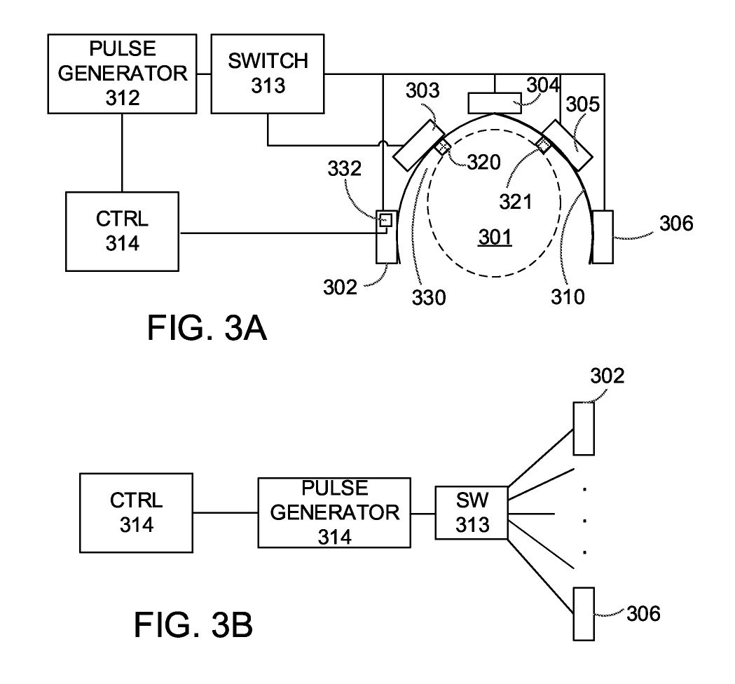

A representative apparatus for applying fields is shown in FIGS. 3A-3B. Coils 302-306 are situated about a target region 301 so that fields can be applied based on an energization of a selected one or more of the coils 302-306. Typically, the coils 302-306 are secured to a hollow frame 310 so that the coils 320-306 can be maintained at a fixed spatial relationship with respect to each other and with respect to the target region 301. The coils 302-306 may or may not contact a specimen situated in the target region 301. A generator 312 selectively couples electrical signals to the coils 302-306 via a switch 313 to produce field distributions in the target region 301. As noted above, the fields are generally configured based on determinations made in a controller 314. In some examples, only a few coils are provided, and are movable with respect to the hollow frame 310 for positioning to target a particular specimen volume.

In the example configuration of FIG. 3A, the frame 310 is spaced apart from the target region with spacers 320, 321. In typical examples, the frame is a rigid shell such as a helmet, and the spacers 320, 321 are made of compliant, thermally insulating materials such as a foam so as to space the frame 310 and coils 302-306 away from a head of a patient. Two separate spacers are shown in FIG. 3A, but more spacers can be used, or spacers such as spacer pads or strips, or a continuous layer of spacer material can be secured to the frame 310. If desired, a volume 330 between the subject's head and the frame 310 can be filled with an insulating material to provide comfort, especially in applications in which the coils 302-306 may generate appreciable heat during operation. Alternatively, the volume 330 can be left open and a fan or other device can be situated to provide a cooling flow of air, and the frame 310 can be provided with cut-outs that permit airflow through the frame 310. In addition, one or more temperature sensors such as temperature sensor 332 can be coupled to the controller 314 so that excessive temperatures are not reached. In some examples, each of the coils 302-306 is provided with a temperature sensor that is coupled to the controller 314.

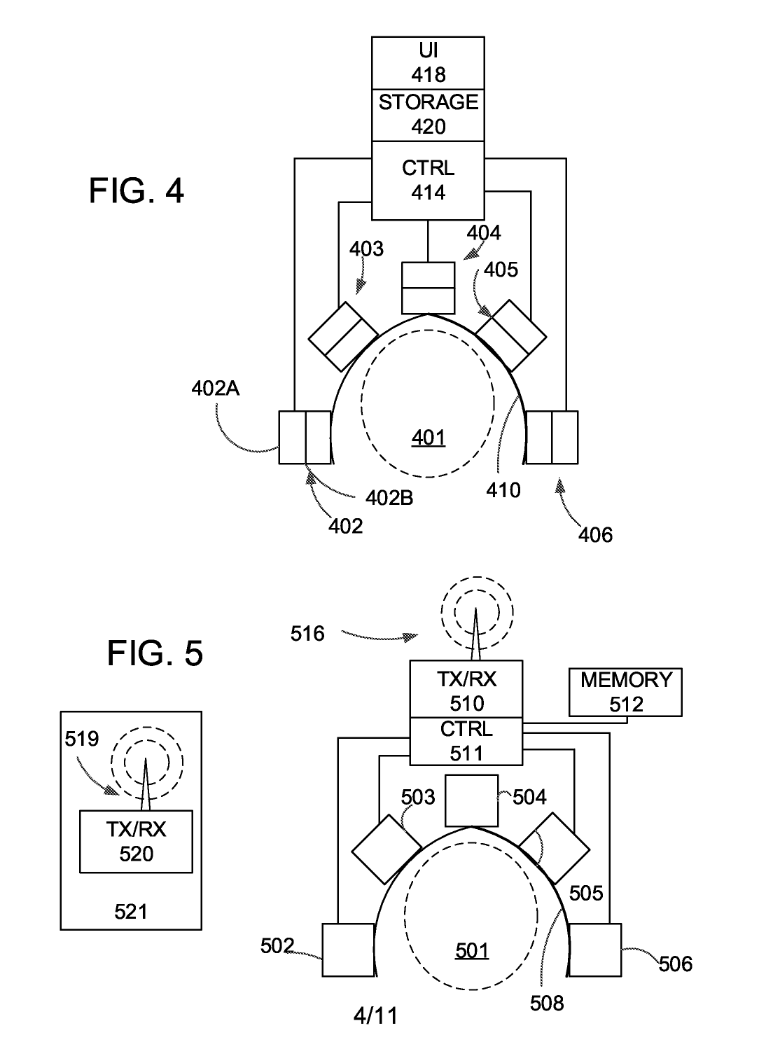

In an alternative arrangement shown in FIG. 4, electromagnetic TTF generating coils 402-406 are secured to a helmet 410. A controller 414 is coupled to the coils 402-406 so as to apply selected sequences or series of waveforms to a specimen situated in a target region 401. The electromagnetic TTF generating system 402 includes a pulse generator 402A and a coil 402B that produces a time varying magnetic field in response to electrical pulses from the pulse generator 402A. With this apparatus, pulse generators and coils can be secured the helmet. If desired, the controller 414 can be secured to the helmet 410 as well. Pulse generators and coils can be configured as described in Boyden et al., U.S. Patent Application Publication 2009/0018384 or, Schneider et al., U.S. Pat. No. 8,523,753, both of which are incorporated herein by reference. In some embodiments, the electromagnetic TTF generating system 402 includes a battery and thus the apparatus can be used without wired power connections. The helmet 410 can include straps, pads, or other features so as to secure the helmet 410 and coils or the electromagnetic TTF generator 402-406 with respect to the target region 401 so as to provide electromagnetic fields to a selected portion of a subject's brain.

Data storage 420 can be provided as, for example, random access or other memory that stores pulse characteristics (duration, field strength, time of application, frequency of application) to be used by the controller 414 in activating electromagnetic TTF generating system. In addition, the data storage can be used to record and confirm application of fields so that a clinician can verify exposures. One or more user interface devices such as switches, displays, touch screens, indicator lights, audible alarms, vibration devices, or other devices that provide visible, audible, or tactile indications of device activity. The storage 420 can be provided as removable storage as a USB memory or a memory card.

In another example shown in FIG. 5, a transceiver 510 is coupled to a controller 511, and both are secured to a helmet 508 that can be fixed to the head of a patient so as to permit exposures to electromagnetic fields within a region 501 by energizing coils 502-506. Treatment field prescriptions and/or pulse sequences can be stored in a memory 512, which is also secured to the helmet 508. An antenna 516 is coupled to the transceiver 510 so as to send and receive signals from an antenna 519 that is coupled to a transceiver 520 of a mobile computing device 521. As shown in FIG. 5, a treatment apparatus is secured to a helmet to form a portable apparatus that can provide lengthy treatments, but still provide patient comfort and mobility.

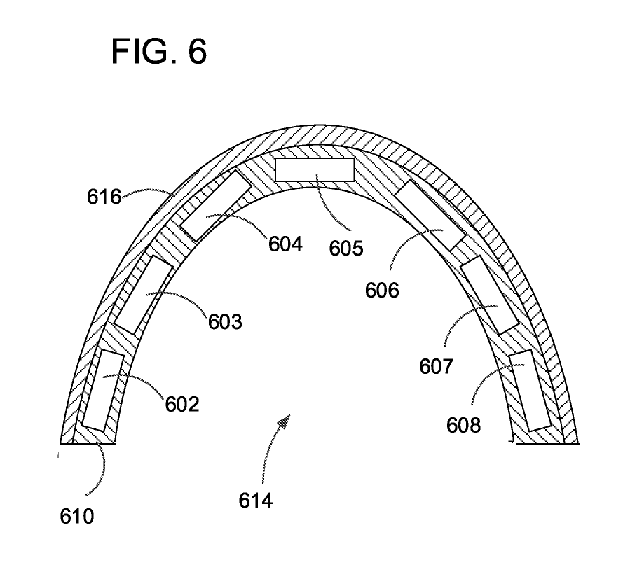

Other mounting configurations can be used to fix a treatment apparatus with respect the head of the patient. Referring to FIG. 6, coils 602-608 are retained in a compliant spacer layer 610 situated about a target space 614. A protective layer 616 or a rigid shell can be secured to the spacer layer 610, if desired. The spacer layer 610 can have a predetermined shape so as to substantially correspond to an average patient head. In still other examples, the coils (and associated circuitry) can be secured to a flexible support such as cloth that can serve as a head covering. Hats of various configurations can be used as well. Such arrangements make application of treatment fields over long durations practical and convenient. For applications to other areas, coils can be secured to or by any form filling garment such as socks, bras, compression shirts, tights, leggings, gloves, headbands, scarves, or other clothing items. In other examples, coils can be provided in cushions or other objects that are not secured to the subject, but which are convenient for exposure, particularly long exposures.

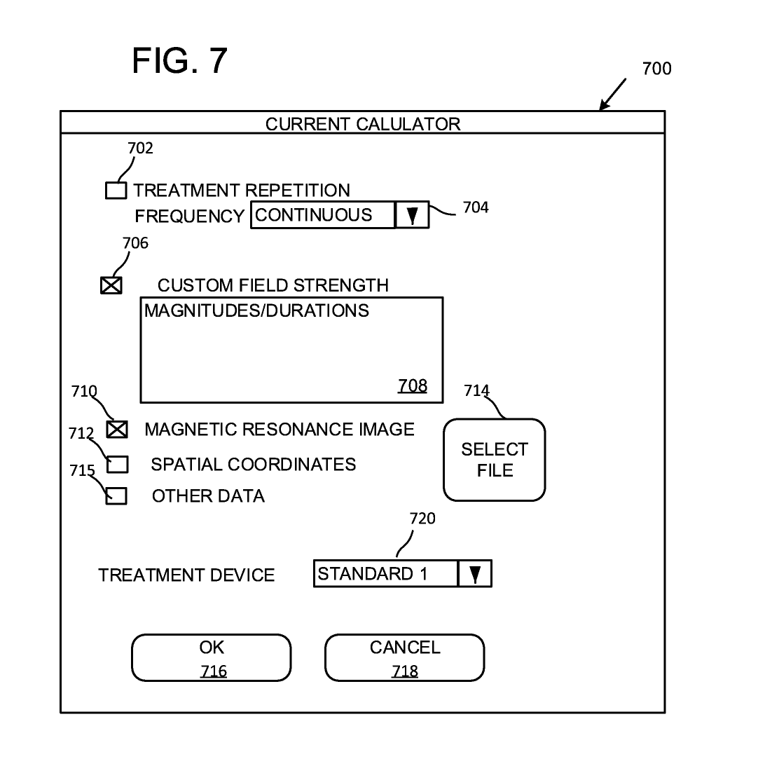

FIG. 7 shows a screen shot 700 of an exemplary user interface for obtaining treatment field generating currents or voltages. A checkbox 702 is provided for selecting an appropriate treatment frequency in a drop down menu 704. As shown, continuous treatment is selected, but treatment repetition can be selected so as to be periodic or aperiodic, and time of day for treatment can also be selected. While default values for treatment fields can be used, custom field strengths can be selected with checkbox 706, and input into a data entry box 708. Spatial location of treatment areas can be obtained from a patient image such as a magnetic resonance image selected using checkbox 710; spatial coordinates based on spatial coordinates or other data can be selected with checkboxes 712, 715, respectively, and any associated data or image files selected for input by clicking a button 714. Data entry can be accepted or rejected with buttons 716, 718. The user interface can also be used to receive externally generated treatment conditions. Based on data input with the user interface, suitable drive levels for treatment field generation are obtained for a treatment device that is selectable with a drop down menu 720. Using MRI and other imaging data, personalized computational electrical models of the brain or other body organs or parts based on an individual's imaging data can be used to specify doses and estimate possible tissue responses to TTFs.

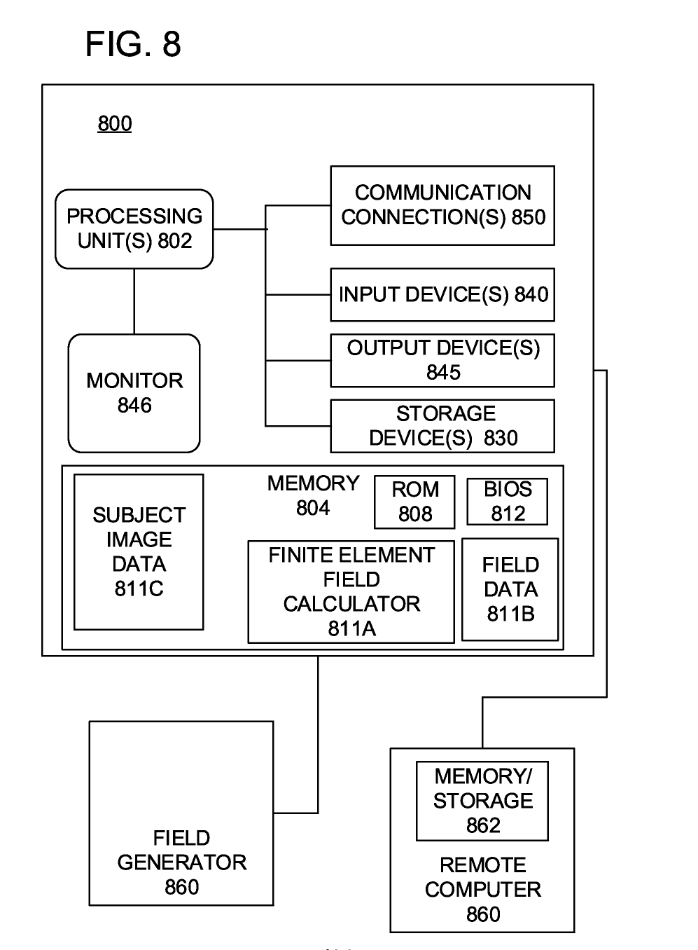

FIG. 8 and the following discussion are intended to provide a brief, general description of an exemplary computing environment in which the disclosed technology may be implemented. Typically, FEM or other electromagnetic field modeling applications are used to establish preferred currents or current sequences that target selected areas for treatment. Although not required, the disclosed technology is described in the general context of computer executable instructions, such as program modules, being executed by a personal computer (PC). Generally, program modules include routines, programs, objects, components, data structures, etc., that perform particular tasks or implement particular abstract data types. Moreover, the disclosed technology may be implemented with other computer system configurations, including hand held devices, multiprocessor systems, microprocessor-based or programmable consumer electronics, network PCs, minicomputers, mainframe computers, and the like. The disclosed technology may also be practiced in distributed computing environments where tasks are performed by remote processing devices that are linked through a communications network. In a distributed computing environment, program modules may be located in both local and remote memory storage devices.

With reference to FIG. 8, an exemplary system for implementing the disclosed technology includes a general purpose computing device in the form of an exemplary conventional PC 800, including one or more processing units 802, a system memory 804, and a system bus 806 that couples various system components including the system memory 804 to the one or more processing units 802. The system bus 806 may be any of several types of bus structures including a memory bus or memory controller, a peripheral bus, and a local bus using any of a variety of bus architectures. The exemplary system memory 804 includes read only memory (ROM) 808 and random access memory (RAM) 810. A basic input/output system (BIOS) 812, containing the basic routines that help with the transfer of information between elements within the PC 800, is stored in ROM 808. The memory 804 also includes a memory portion 811A that stores computer-executable instructions for computing coil currents or other field stimuli so as to achieve effective field strengths at target areas. The resulting field data can be stored in the memory 804 at memory portion 811B. The field data can also include prescribed exposure conditions for use by the field calculator. In some examples, field calculations are based on individual subject images or image data stored in memory 811C or obtained over a local area or wide area network. Such field calculations can include tissue inhomogeneity, tissue boundaries, tissue orientations, and other features that are common to all subjects or specific to an individual subject. One example of such computations is described in Miranda et al., “Tissue heterogeneity as a mechanism for localized neural stimulation by applied electric fields,” Phys. Med. Biol. 52:5603-5617 (2007). These individualized field calculations can be performed at various network locations, or by the device used to apply TTFs.

The magnitude, direction and distribution of induced electric fields in a tissue can be important determinants of treatment efficacy. These field characteristics can be estimated based on image data or other patient-specific data. For example, for treatment of brain cancer, a numerical head model can be created from one or more MRIs. Voxel size can be selected as needed, and in one example, a voxel size of 1 mm³ is selected. MRIs can be segmented into different tissue types such as scalp, skull, cerebrospinal fluid (CSF), gray matter (GM), and white matter (WM). In some cases, data for head models can be obtained with electrode arrays or pairs of such arrays placed on the scalp, and currents can be applied to the electrodes with a selected frequency and amplitude, such as at about 200 kHz and 100 mA. The magnitude of the electric field is typically higher in white matter because its impedance is higher than that of gray matter. The low impedance of CSF in the ventricles also affects the electric field in nearby brain tissue. The electric field is not uniform as it is affected by the distribution of tissue types, the location and orientation of interfaces between them, and their individual electrical properties. As a result of tissue heterogeneity, shunting as well as concentration of current and electric field can be observed in different parts of the brain. The inclusion of anisotropy in the electrical conductivity of white matter in models can be used to further quantify shunting and spatial non-uniformity.

In some examples, induced fields having time-varying directions and at two or more frequencies can be used. For example, a field direction can vary during a single field application or vary from exposure to exposure. Each field direction can be associated with a different field magnitude, if desired. Similarly, a single field application can have a plurality of frequency components, or a series of different frequencies can be applied.

The exemplary PC 800 further includes one or more storage devices 830 such as a hard disk drive for reading from and writing to a hard disk, a magnetic disk drive for reading from or writing to a removable magnetic disk, and an optical disk drive for reading from or writing to a removable optical disk (such as a CD-ROM or other optical media). Such storage devices can be connected to the system bus 806 by a hard disk drive interface, a magnetic disk drive interface, and an optical drive interface, respectively. The drives and their associated computer readable media provide nonvolatile storage of computer-readable instructions, data structures, program modules, and other data for the PC 800. Other types of computer-readable media which can store data that is accessible by a PC, such as magnetic cassettes, flash memory cards, digital video disks, CDs, DVDs, RAMs, ROMs, and the like, may also be used in the exemplary operating environment.

A number of program modules may be stored in the storage devices 830 including an operating system, one or more application programs, other program modules, and program data. In some examples, electromagnetic field calculation applications such as finite element method calculators are stored in the storage device 830. A user may enter commands and information into the PC 800 through one or more input devices 840 such as a keyboard and a pointing device such as a mouse. Other input devices may include a digital camera, microphone, joystick, game pad, satellite dish, scanner, or the like. These and other input devices are often connected to the one or more processing units 802 through a serial port interface that is coupled to the system bus 806, but may be connected by other interfaces such as a parallel port, game port, or universal serial bus (USB). A monitor 846 or other type of display device is also connected to the system bus 806 via an interface, such as a video adapter. Other peripheral output devices, such as speakers and printers (not shown), may be included.

The PC 800 may operate in a networked environment using logical connections to one or more remote computers, such as a remote computer 860. In some examples, one or more network or communication connections 850 are included. The remote computer 860 may be another PC, a server, a router, a network PC, or a peer device or other common network node, and typically includes many or all of the elements described above relative to the PC 800, although only a memory storage device 862 has been illustrated in FIG. 8. The personal computer 800 and/or the remote computer 860 can be connected to a logical a local area network (LAN) and a wide area network (WAN). Such networking environments are commonplace in offices, enterprise wide computer networks, intranets, and the Internet. A field generator 860 (such as coils and associated drive circuitry) can be coupled to the PC 800, but in many cases, the field generator 860 is activated according to instructions produced using the field calculator, but is not connected to the PC 800.

When used in a LAN networking environment, the PC 800 is connected to the LAN through a network interface. When used in a WAN networking environment, the PC 800 typically includes a modem or other means for establishing communications over the WAN, such as the Internet. In a networked environment, program modules depicted relative to the personal computer 800, or portions thereof, may be stored in the remote memory storage device or other locations on the LAN or WAN. The network connections shown are exemplary, and other means of establishing a communications link between the computers may be used.

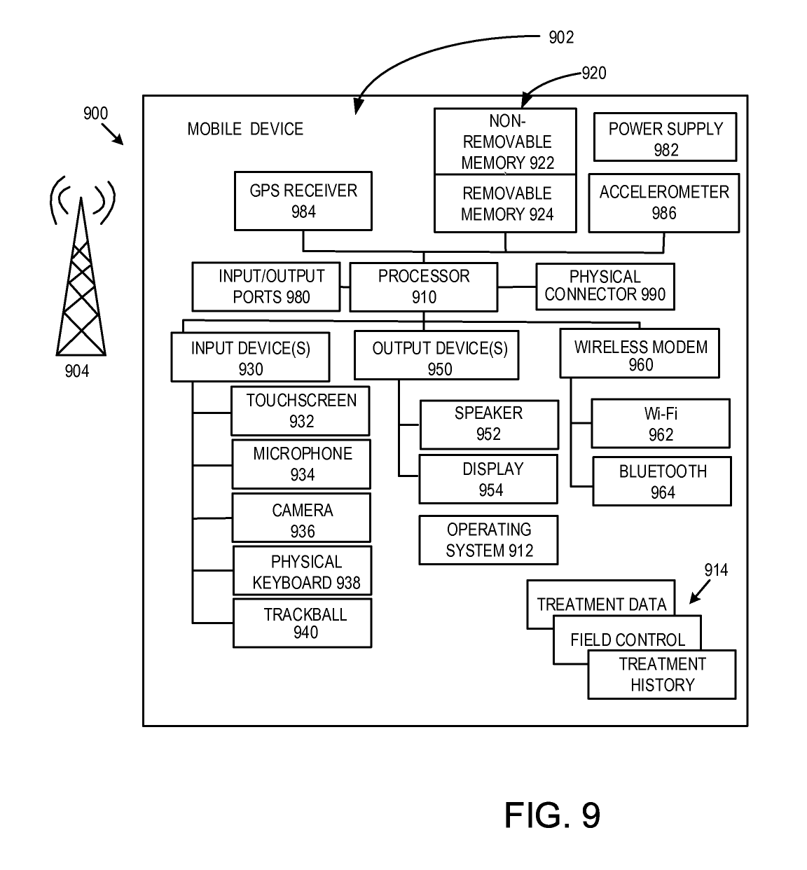

FIG.9 is a system diagram depicting an exemplary mobile device 900 including a variety of optional hardware and software components, shown generally at 902. Any components 902 in the mobile device can communicate with any other component, although not all connections are shown, for ease of illustration. The mobile device can be any of a variety of computing devices (e.g., cell phone, smartphone, handheld computer, Personal Digital Assistant (PDA), etc.) and can allow wireless two-way communications with one or more mobile communications networks 904, such as a cellular or satellite network.

The illustrated mobile device 900 can include a controller or processor 910 (e.g., signal processor, microprocessor, ASIC, or other control and processing logic circuitry) for performing such tasks as signal coding, data processing, input/output processing, power control, and/or other functions. An operating system 912 can control the allocation and usage of the components 902 and support for one or more application programs 914. As shown in FIG. 9, the application programs include applications that receive treatment data from a clinician, compute drive levels required for treatment, perform FEM or other modeling of volumes to be treated, and record treatment history. The application programs can also include common mobile computing applications (e.g., email applications, calendars, contact managers, web browsers, messaging applications), or any other computing application to be used in conjunction with treatment applications to send and receive treatment prescriptions, field calculations, drive levels, and confirmations of treatment status.

The illustrated mobile device 900 can include memory 920. Memory 920 can include non-removable memory 922 and/or removable memory 924. The non-removable memory 922 can include RAM, ROM, flash memory, a hard disk, or other well-known memory storage technologies. The removable memory 924 can include flash memory or a Subscriber Identity Module (SIM) card, which is well known in GSM communication systems, or other well-known memory storage technologies, such as “smart cards.” The memory 920 can be used for storing data and/or code for running the operating system 912 and the applications 914. Example data can include web pages, text, images, sound files, video data, or other data sets to be sent to and/or received from one or more network servers or other devices via one or more wired or wireless networks. The memory 920 can be used to store a subscriber identifier, such as an International Mobile Subscriber Identity (IMSI), and an equipment identifier, such as an International Mobile Equipment Identifier (IMEI). Such identifiers can be transmitted to a network server to identify users and equipment. Treatment prescriptions can be stored in the memory 920.

The mobile device 900 can support one or more input devices 930, such as a touchscreen 932, microphone 934, camera 936, physical keyboard 938 and/or trackball 940 and one or more output devices 950, such as a speaker 952 and a display 954. Other possible output devices (not shown) can include piezoelectric or other haptic output devices. Some devices can serve more than one input/output function. For example, touchscreen 932 and display 954 can be combined in a single input/output device. The input devices 930 can include a Natural User Interface (NUI). An NUI is any interface technology that enables a user to interact with a device in a “natural” manner, free from artificial constraints imposed by input devices such as mice, keyboards, remote controls, and the like. Examples of NUI methods include those relying on speech recognition, touch and stylus recognition, gesture recognition both on screen and adjacent to the screen, air gestures, head and eye tracking, voice and speech, vision, touch, gestures, and machine intelligence. Other examples of a NUI include motion gesture detection using accelerometers/gyroscopes, facial recognition, 3D displays, head, eye, and gaze tracking, immersive augmented reality and virtual reality systems, all of which provide a more natural interface, as well as technologies for sensing brain activity using electric field sensing electrodes (EEG and related methods). Thus, in one specific example, the operating system 912 or applications 914 can comprise speech-recognition software as part of a voice user interface that allows a user to operate the device 900 via voice commands. Further, the device 900 can comprise input devices and software that allows for user interaction via a user's spatial gestures.

A wireless modem 960 can be coupled to an antenna (not shown) and can support two-way communications between the processor 910 and external devices, as is well understood in the art. The modem 960 is shown generically and can include a cellular modem for communicating with the mobile communication network 904 and/or other radio-based modems (e.g., Bluetooth 964 or Wi-Fi 962). The wireless modem 960 is typically configured for communication with one or more cellular networks, such as a GSM network for data and voice communications within a single cellular network, between cellular networks, or between the mobile device and a public switched telephone network (PSTN).

The mobile device can further include at least one input/output port 980, a power supply 982, a satellite navigation system receiver 984, such as a Global Positioning System (GPS) receiver, an accelerometer 986, and/or a physical connector 990, which can be a USB port, IEEE 1394 (FireWire) port, and/or RS-232 port. The illustrated components 902 are not required or all-inclusive, as any components can be deleted and other components can be added.

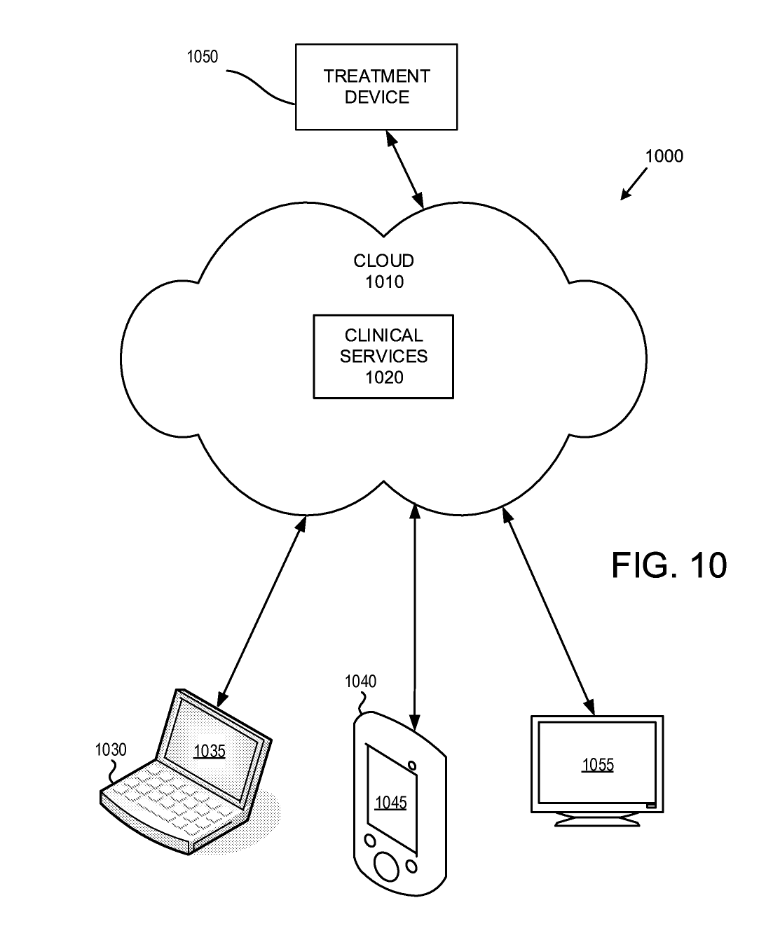

FIG. 10 illustrates a generalized example of a suitable implementation environment 1000 in which described embodiments, techniques, and technologies may be implemented. In example environment 1000, various types of services (e.g., computing services) are provided by a cloud 1010. For example, the cloud 1010 can comprise a collection of computing devices, which may be located centrally or distributed, that provide cloud-based services to various types of users and devices connected via a network such as the Internet. These devices can be coupled to diagnostic devices such as magnetic resonance imaging devices, finite element field and current calculators, as well as treatment devices and clinician record keeping systems. The implementation environment 1000 can be used in different ways to accomplish computing tasks. For example, some tasks (e.g., processing user input and presenting a user interface) can be performed on local computing devices (e.g., connected devices 1030, 1040, 1050) while other tasks (e.g., storage of data to be used in subsequent processing) can be performed in the cloud 1010. For example, connected device 1040 can be used to provide instructions to a treatment device 1050.

In example environment 1000, the cloud 1010 provides services for connected devices 1030, 1040, 1050 with a variety of screen capabilities. Connected device 1030 represents a device with a computer screen 1035 (e.g., a mid-size screen). For example, connected device 1030 could be a personal computer such as desktop computer, laptop, notebook, netbook, or the like. Connected device 1040 represents a device with a mobile device screen 1045 (e.g., a small size screen). For example, connected device 1040 could be a mobile phone, smart phone, personal digital assistant, tablet computer, or the like. Connected device 1050 represents a device with a large screen 1055. For example, connected device 1050 could be a television screen (e.g., a smart television) or another device connected to a television (e.g., a set-top box or the like. One or more of the connected devices 1030, 1040, 1050 can include touchscreen capabilities. Touchscreens can accept input in different ways. For example, capacitive touchscreens detect touch input when an object (e.g., a fingertip or stylus) distorts or interrupts an electrical current running across the surface. As another example, touchscreens can use optical sensors to detect touch input when beams from the optical sensors are interrupted. Physical contact with the surface of the screen is not necessary for input to be detected by some touchscreens. Devices without screen capabilities also can be used in example environment 1000. For example, the cloud 1010 can provide services for one or more computers (e.g., server computers) without displays.

Services can be provided by the cloud 1010 through service providers such as a clinical services provider 1020, or through other providers of online services (not depicted). For example, cloud services can be customized to the screen size, display capability, and/or touchscreen capability of a particular connected device (e.g., connected devices 1030, 1040, 1050) for communication with treating physicians.

In some cases, individualized electric field distributions are determined for a particular treatment based on one or more electrical characteristics of a region to be treated. Such distributions can be based on specimen image such as magnetic resonance imaging or computed tomography. Tissue properties in a region to be treated can be characterized to estimate conductivity, dielectric constants, and frequency response. Then, exposures can be personalized as to duration, orientation, magnitude, and frequency.

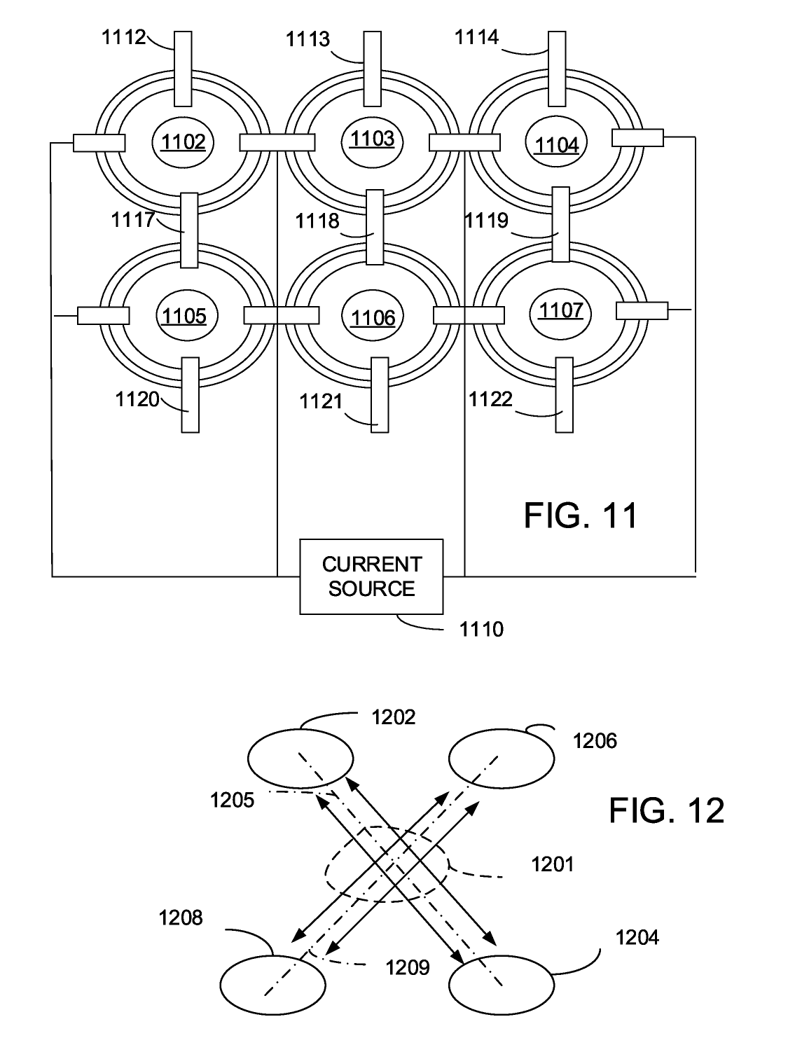

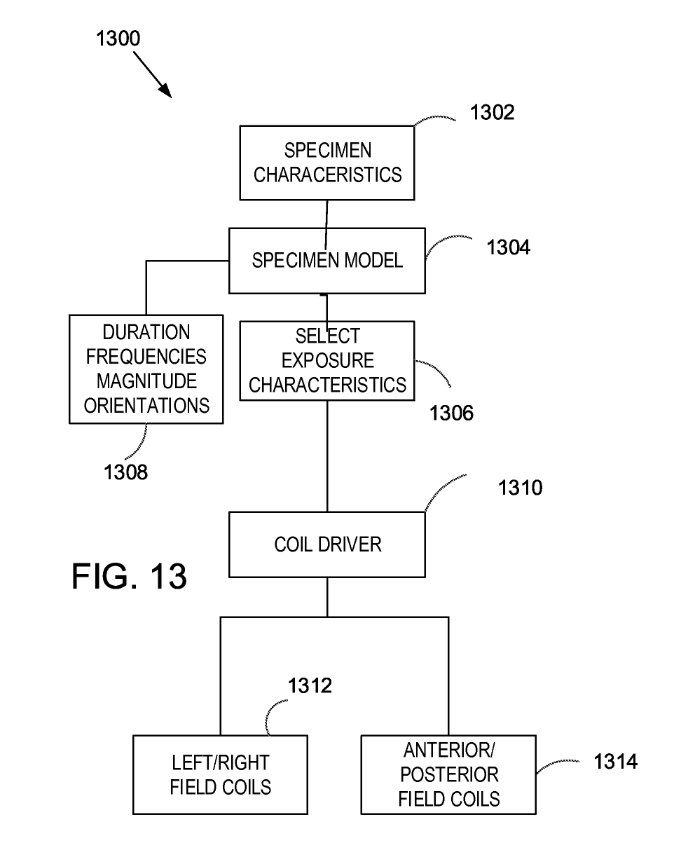

FIG. 11 illustrates a representative coil assembly 1100 that includes a plurality of coils 1102-1107 that are electrically coupled to a current source 1110. The coils 1102-1107 can be rigidly or flexibly coupled with connectors 1112-1122 that can also serve as electrical connectors or including switches or switching circuit to permit selection of particular coils to be energized. Additional coils and coils of difference sizes and shapes can be used. As shown in FIG. 11, the coils 1102-1107 include current paths having different diameters so that areas associated with magnetic field variations can be selected. The coils 1102-1107 can have selectable diameters or can include one or more subcoils. Coils can define rectangular, polygonal, oval, elliptical, circular, or other current paths that comprise one or more straight line or curved segments. The coils assembly 1100 can include coils arranged for exposure of a selected region to time-varying magnetic fields to produce induced electric fields. Different regions of a subject can be exposed (e.g., arms, head, torso, ankles, wrists, legs) or coils can be arranged for exposure of various organs or tissues (e.g., brain, stomach, lungs). In one example, coils can be associated with selected hexagons and pentagons of a truncated icosahedron to enclose or partially enclose a specimen volume. A soccer ball coil configuration for use in magnetic resonance imaging is described in Wiggins et al., “32-channel 3 Tesla receive-only phased-array head coil with soccer-ball element geometry,” Magn. Res. Med. 56:216-23 (2006). In still other examples, smaller numbers of coils can be used, and situated to produce induced electric fields so as to target particular regions. Current waveforms and amplitudes could be controlled individually for each coil.

Application of induced fields may be more effective if the induced fields are suitably aligned. Absent a known, predetermined alignment of a specimen or subject region to be exposed, applied field directions can be varied as illustrated schematically in FIG. 12. Coils 1202, 1204 are situated to produce magnetic fields in a specimen volume 1201 along a first axis 1205, such as an anterior-posterior axis. Coils 1206, 1208 are situated to produce magnetic fields in the specimen volume 1201 along a second axis 1209, such as a left-right axis. Additional coils and axes can be used, and varying directions can be achieved with multiple coils or coil sets, or by adjusting a relative orientation of the specimen volume 1201 with respect to one or more coils.

One or more coils can be energized at a single frequency or a swept frequency to produce induced fields at a single frequency or multiple frequencies. Coils can be arranged to induce electric fields in two or more directions simultaneously although care must generally be taken so that multiple induced fields do not tend to cancel.

A representative TTF exposure method 1300 includes storing a set of specimen characteristics at 1302. Such as electrical and structural characteristics can be obtained by specimen imaging or other processes. A specimen model is obtained at 1304 and suitable exposure characteristics are selected at 1306. Various characteristics can be selected from a set of exposure values stored at 1308. Corresponding currents are selected at 1310 and delivered to coils sets such as left/right coils 1312 and anterior/posterior coils 1314.

In view of the many possible embodiments to which the principles of the disclosed technology may be applied, it should be recognized that the illustrated embodiments are only preferred examples and should not be taken as limiting the scope of the invention. We therefore claim as our invention all that comes within the scope and spirit of these claims.

1. A system, comprising:
a plurality of coils; and
an electromagnetic field generator that energizes the plurality of coils so as to apply a tumor treating field (TTF) to a specimen region, wherein the TTF has a time varying magnetic field component that induces an electric field of magnitude of at least 0.1 V/cm at a frequency of between 50 kHz and 500 kHz.

2. The system of claim 1, wherein the specimen region is associated with a tumor.

3. The system of claim 2, wherein the tissue region is associated with a brain tumor.

4. The system of claim 1, wherein the electromagnetic field generator energizes the plurality of coils so as to apply the TTF to the specimen region along a plurality of axes.

5. The system of claim 1, wherein the electromagnetic field generator sequentially energizes the plurality of coils so as to sequentially apply the TTF to the specimen region along at least two axes.

6. The system of claim 5, wherein the electromagnetic field generator energizes the plurality of coils so that the TTF is applied at a plurality of frequencies between 50 kHz and 500 kHz.

7. The system of claim 1, further comprising a rigid shell that is shaped to at least partially enclose the specimen region, wherein the plurality of coils is secured to the rigid shell.

8. The system of claim 1, wherein the rigid shell is a portion of a helmet.

9. The system of claim 1, wherein the coils of the plurality of coils are flexibly interconnected so as to be wrappable about the specimen region.

10. The system of claim 9, wherein coils are energized by the electromagnetic field generator so as to produce a time-varying magnetic field that produces an induced electric field magnitude of at least 0.5, 1.0, 2, 5, 10, or 100 V/cm.

11. A method, comprising:
identifying a tissue to be exposed to a treatment electromagnetic field; and
applying the treatment electromagnetic field to a portion of the tissue, the treatment electromagnetic field having a time-varying magnetic field component that produces an induced electric field having an effective magnitude of at least 0.5 V/cm at a frequency of between about 50 kHz and 500 Hz in the region to be exposed.

12. The method of claim 11, further comprising determining the applied treatment electromagnetic field based on electrical characteristics of the tissue determined from an image of the tissue.

13. The method of claim 11, wherein the treatment electromagnetic field is applied along at least two axes.

14. The method of claim 13, wherein the tissue is a portion of a patient brain.

15. The method of claim 14, further comprising applying the treatment electromagnetic field along a plurality of axes.

16. The method of claim 15 further comprising applying the treatment electromagnetic field at a plurality of frequencies in the range of 50 kHz to 500 kHz.

17. The method of claim 14, wherein the treatment electromagnetic field is applied so as to have an effective duration of at least 10 ms, 100 ms, 1 s, 10 s, 100 s , or 1000 s.

18. The method of claim 11, wherein the treatment electromagnetic field is applied with coils that are fixed with respect to the tissue.

19. The method of claim 11, further comprising applying the treatment electromagnetic field along different axes by selectively energizing corresponding coils.

20. The method of claim 11, further comprising determining the treatment electromagnetic field based on an electrical model of the tissue that includes at least one tissue permittivity and at least one tissue conductivity.

21. At least one non-transitory computer readable medium comprising computer-executable instructions for a method of applying treatment electromagnetic fields to a tissue, the method comprising:
selecting a portion of the tissue exhibiting cancer;
generating a sequence of electrical currents and coupling the sequence of electrical currents to at least one coil so as to produce the treatment electromagnetic fields in the selected portion of the tissue, wherein the treatment electromagnetic field includes a time-varying magnetic field that produces an electric field having a magnitude of at least 0.1 V/cm.

22. The at least one computer readable medium of claim 21, wherein the electrical currents have magnitudes such that the treatment electromagnetic fields have time varying magnetic field components that produce an induced electric field magnitude of at least 0.5, 1.0, 2, 5, 10, or 100 V/cm.

23. The at least one computer readable medium of claim 21, wherein the treatment electromagnetic fields are applied along a least two axes.

24. The at least one computer readable medium of claim 21, wherein the electrical currents are selected based on a sequence of electrical currents includes electrical currents at least two frequencies between 50 kHz and 1 MHz.

25. The at least one computer readable medium of claim 23, wherein the selected portion of the brain is identified as exhibiting GBM.

26. The at least one computer-readable medium of claim 21, wherein the method further comprises estimating an electromagnetic field distribution in the tissue based on a tissue image and at least one tissue permittivity and at least one tissue conductivity, wherein the sequence of electrical currents is based on the estimated electromagnetic field distribution.

27. The at least one computer-readable medium of claim 26, wherein the tissue image is an MR image.

28. The method of claim 11, wherein the tissue comprises a Glioblastoma Multiforme brain tumor.
